(19) United States
(12) Patent Application Publication
(10) Pub. No.: US 2009/0187128 A1
(43) Pub. Date: Jul. 23, 2009

(54) Shoulder-neck-shaper

(76) Inventor: Siddappa Nagabhushan, Frostburg, MD (US)

Correspondence Address: SIDDAPPA NAGABHUSHAN, 16327 Harwood drive SE, Frostburg, MD, 21532, US

(21) Appl. No.: 11/978,506

(22) Filed: Jan. 22, 2008

(51) Int. Cl. A61F 5/00; A63B 23/025

(52) U.S. Cl. 602/18; 602/19; 482/10

(57) ABSTRACT

Shoulder-Neck-Shaper is designed mainly to alleviate the discomfort of upper back and neck in workers.

Since the revolution of “IT” and “service industries” more and more of the workers sit at the desk hours and hours with neck bent and shoulder drooping forwards.

Similarly workers working over the counter tops have similar complaints. SNS unit will help in squaring the shoulders relieving the constant stretch of upper back of the top and inner connecting muscles of the shoulder blades.

Similarly a more natural position of the neck will help to relieve the discomfort at the junction of neck and upper thoracic spine. It may also relieve any pins and needles in arms by opening numeral foramina of the neck bones by relieving any stretched nerves of neck and upper back.

### BACKGROUND OF THE INVENTION

The invention relates to a shoulder neck shaping. In particular, the invention is shoulder neck shaper that is adapted to be used as medical equipment to improve posture. The equipment works the muscles that improve posture.

Ever since we stand up and walk, our entire body is prone to fall forward by the pull of gravity. As we age, this pull can result in drooped head and rounded shoulders.

Most of the occupations like, Desk jobs, Secretaries, Receptionists, Sales people who work over the counters may have severe needling pain, tight muscle pull between the shoulder blades as well as neck pain, tension head aches and usually arm pains.

Exercises by shoulder-neck-shaper consist of realigning the drooped head and working on rounded shoulders to make it square.

Shoulder and neck are connected by top big trapeziums and many smaller flat muscles that are deeper and hold neck and shoulder blades together as well as between the shoulder blades.

Shoulder neck shaper works exactly on the principle of working on shoulder blades and head and neck muscles together at the same time.

Working individually, upper back, head and neck may not solve the problem since muscles connecting upper back, neck and shoulder at the same. These muscles may simply yield at different ends with out helping the problems.

A fully aligned head and neck as well square shoulder will improve your posture and relax your tight upper back muscles.

A normally aligned neck will open up “neural foramina” to improve exiting nerves to your neck and upper back to under less or no tension to ease your neurological problems to head and neck and to your arms.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a perspective view of the chin support

FIG. 2 is a perspective view of the vertical height adjuster.

FIG. 3 is a perspective view of the vertical extension elastic spring with a steel ring.

FIG. 4 is a perspective view of the anchoring base.

FIG. 5 is a perspective view of the shoulder neck shaper assembled.

FIG. 6 is a perspective view of the instrument used by a user.

### DETAILED DESCRIPTION OF THE PREFERRED EMBODIMENTS

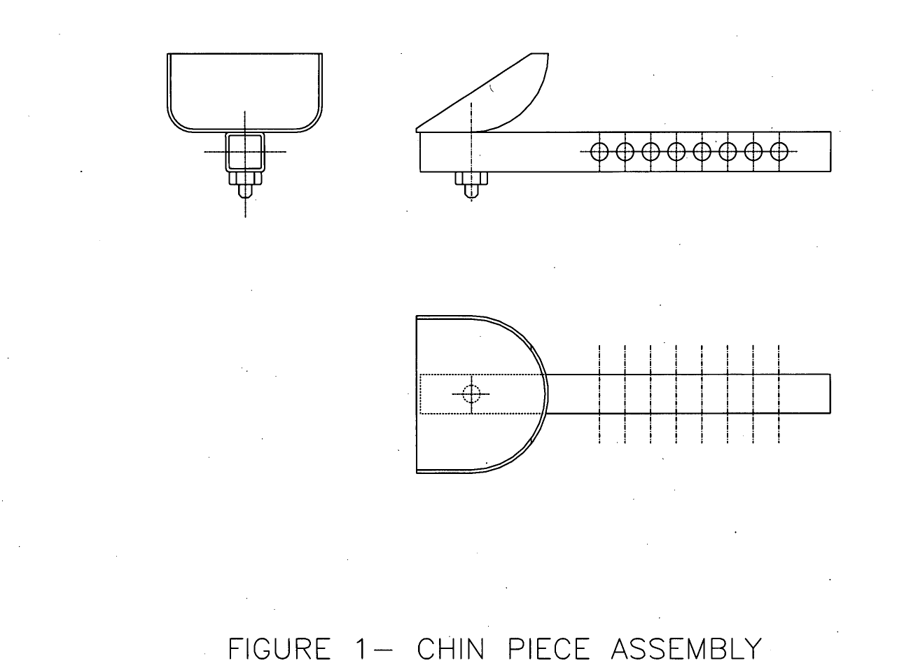

FIG. 1 illustrates a chin support with multiple through and through holes is punched at half-inch increments at the side of the bar. The chin support is a plastic chin support that is screwed in at the very end of the bar facing towards your chin to hold the chin in place.

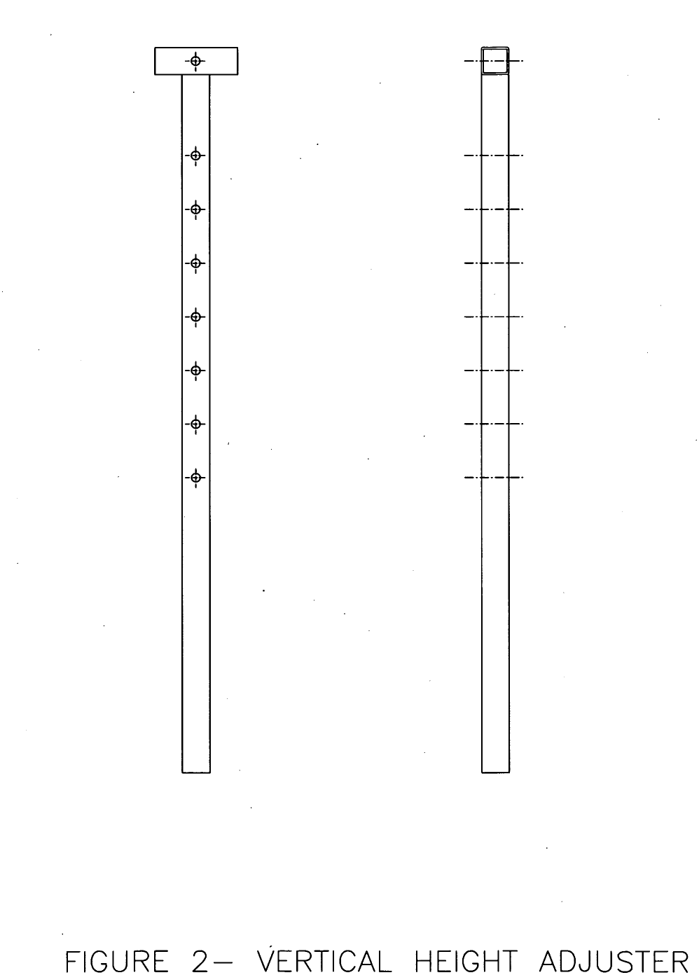

FIG. 2 illustrates a vertical height adjuster that is an 18 inches long tube with multiple holes through and through and side to side at ½ inch increments. A tube 2 inches in length is welded on top of the Vertical Height Adjuster in parallel to base unit. A cavity within the aluminum tube is facing towards the user and at the side of the multiple holes previously made.

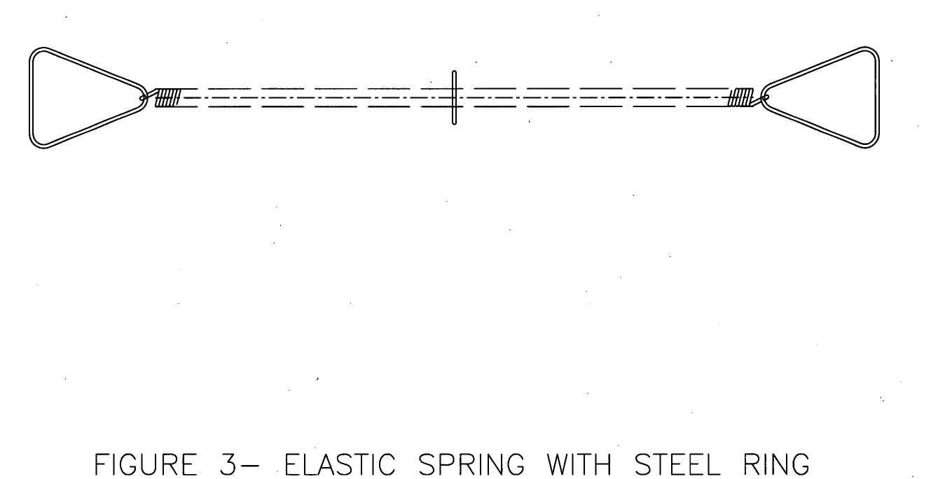

FIG. 3 illustrates a vertical extension elastic spring with a steel ring that is an elastic spring of stainless steel measuring 18 inches in length with a thickness of 1.5 to 2 mm width. There is an out flared aluminum handles on both ends. There is a 1 inch diameter of steel ring with the thickness of 2-3 mm is passed over the elastic spring. On both ends of the elastic springs with circled out flares are hooked to an aluminum handle.

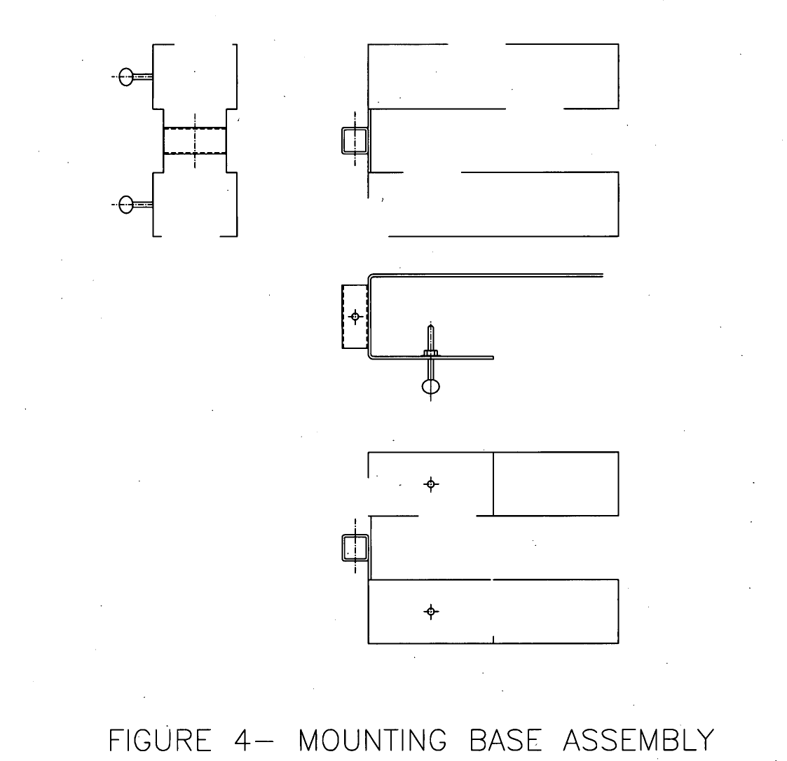

FIG. 4 illustrates an anchoring base is made of thick flat non malleable aluminum sheet with A two inch wide and 10 inch long strip is cut and bent at a right angle at 6 inch and 4 inch length. A 6 inch long and 2 inch wide strip are welded between the above two strips in the middle of 2 inch width without any bend and being flat in one piece. 2 to 4 screw-drill holes are made on 4 inch strip to anchor to the top of the table or on a suitable flat surface with a suitable thickness and held firmly with butterfly wing screws. Metal tubes with a length of two inches are welded on to a 6 inch strip in the middle, facing the user without any tilt and parallel to the aluminum strip. The cavity is orientated vertically at right angles to the strip. All sharp ends are rounded off except the cavities of the vertical height adjuster and anchoring base.

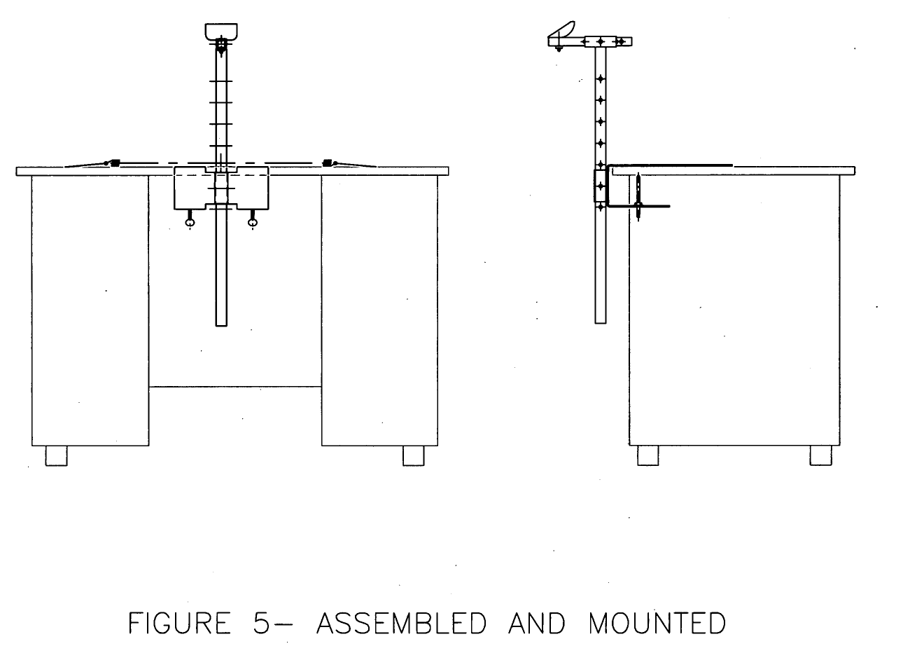

FIG. 5 illustrates the shoulder neck shaper assembled.

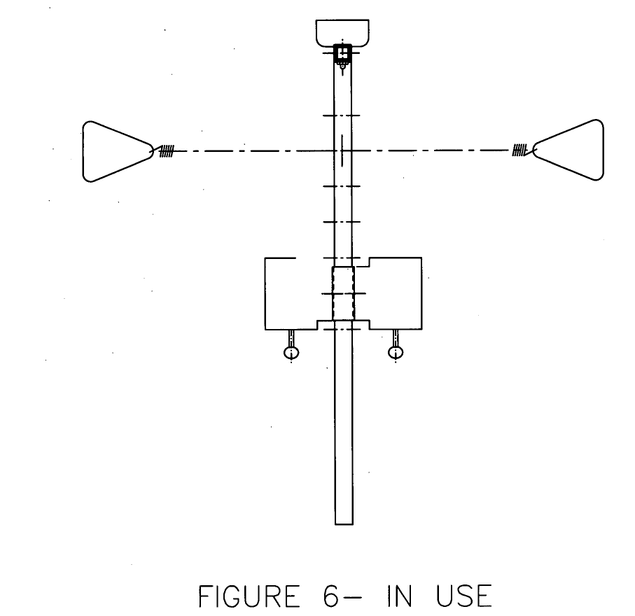

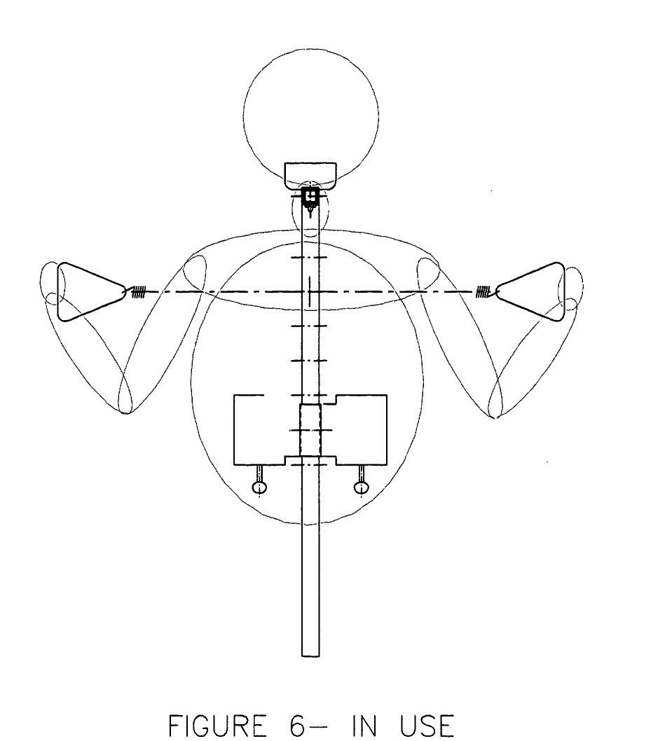

FIG. 6 illustrates the shoulder neck shaper instrument used by a user.

In conclusion, herein is presented a shoulder neck shaper. The invention is illustrated by example in the drawing figures, and throughout the written description. It should be understood that numerous variations are possible, while adhering to the inventive concept. Such variations are contemplated as being a part of the present invention.

What is claimed is:

1. A shoulder neck shaper for improving posture of one's body comprising: a chin support, a vertical height adjuster, the metal elastic extension spring with metal ring, and an anchoring base to accommodate the equipment being in a fixed position; a plurality of holds for securing the medical equipment to a hard; wherein the vertical height adjuster comprises an adjustment mechanism for altering the length of the chin support and distributing the weight of the user, and wherein the locks comprise a pair of screws for securing the equipment to the anchoring base, each equipment screw positioned near one of the anchoring base opposed ends for holding the equipment securely in place.

2. The shoulder neck shaper medical equipment as recited in claim 1, wherein the chin support consists of multiple through and through holes in half-inch increments supporting a cushioned plastic chin support.

3. The shoulder neck shaper medical equipment as recited in claim 2, wherein the vertical height adjuster comprises 18 inches metal tube with multiple holes through and through side to side at ½ inch increments, with a tube 2 inches in length is welded on top of the vertical height adjuster to connect chin support.

4. The shoulder neck shaper medical equipment as recited in claim 3, wherein the extension elastic spring with a steel ring comprises of an elastic spring of stainless steel measuring 18 inches in length with an out flared aluminum handles on both ends. Both ends of the elastic springs have circled our flares are hooked to aluminum handles.

5. The shoulder neck shaper medical equipment as recited in claim 4, wherein the anchoring base is made of thick flat non malleable aluminum sheet which are formed into two inch wide and 10 inch long strip is cut and bent at a right angle; consists of wide metal strips that are welded between the right angle strips in the middle having screw drill holes that are made to anchor to the top of a firm board.
